(19) United States
(12) Patent Application Publication
(10) Pub. No.: US 2012/0078163 A1
(43) Pub. Date: Mar. 29, 2012

(54) METHOD OF GENERATING CONNECTIVE TISSUE

(75) Inventors: Dan Gazit, Maccabim (IL); Gadi Pelled, Rishon-LeZion (IL); Zulma Gazit, Maccabim (IL); Nadav Kimelman-Bleich, Jerusalem (IL)

(73) Assignee: Yissum Research Development Company of the Hebrew University of Jerusalem Ltd., Jerusalem (IL)

(21) Appl. No.: 13/322,457

(22) PCT Filed: May 27, 2010
(86) PCT No.: PCT/IL10/00421

§ 371 Date: Nov. 24, 2011

Related U.S. Application Data
(60) Provisional application No. 61/181,371, filed on May 27, 2009.

(51) Int. Cl. A61M 37/00; A61P 19/00; A61K 35/12

(52) U.S. Cl. 604/21; 604/20; 604/22; 424/93.7

(57) ABSTRACT

A method of generating bone or connective tissue in a subject in need thereof, the method comprising: (a) implanting a scaffold in a damaged region of the subject, the scaffold comprising a tissue growth promoting agent; and (b) enhancing uptake of the tissue growth promoting agent into cells located on the scaffold, wherein the enhancing is effected by induced poration of the cells thereby generating bone or connective tissue in the subject. Kits for generating bone or connective tissue are also provided.

### FIELD AND BACKGROUND OF THE INVENTION

The present invention, in some embodiments thereof, relates to methods of generating connective tissue and, more particularly, but not exclusively, to methods of generating bone tissue using a combination of physical DNA transfer of osteogenic genes and implantation of scaffolds.

Multiple fractures and nonunion fractures pose major challenges to orthopedics, for which thus far no optimal solution has been identified. More than 20% of bone fractures heal unsuccessfully. Although autologous bone grafts are considered gold-standard treatment for such conditions, their use can result in donor site morbidity. Recombinant human BMP (rhBMP)-2 and rhBMP-7 are presently the only biological alternatives to bone harvesting. The administration of rhBMPs yields good results (for example, a 44% reduction in the tibia's failure to heal when rhBMP-2 was administrated on a collagen sponge); however, this treatment requires megadoses of the protein (as high as 1.5 mg protein/ml matrix).

To offer a biological alternative for rhBMP-based therapies, many researchers have used genetically engineered stem cells to treat nonunion bone defects in various animal models, from rodents to goats. Induction of bone formation by direct introduction of an osteogenic gene into target tissue has been explored in some studies. In most instances, viral vectors (mainly adenoviral ones) were used, validating the finding that only transient transgene expression is required to induce bone formation. Although use of viruses is more efficient for gene delivery, nonviral gene delivery is sufficient for bone formation.

One nonviral gene delivery method used for bone formation is electrical field-mediated gene transfer, better known as DNA electrotransfer or electroporation: the use of short high-voltage pulses to overcome the barrier of the cell membrane. This method has been successful when applied both in vitro (in vitro electroporation of cells followed by cell implantation) and in vivo (electroporation of cells in situ) [Kawai, M., et al., Hum Gene Ther, 2003. 14(16): p. 1547-56; Kishimoto, K. N., et al., Bone, 2002. 31(2): p. 340-7; Aslan, H., et al., Tissue Eng, 2006. 12(4): p. 877-89]. The electrical pulse destabilizes the cell membrane for a short time, during which small molecules can enter the cell by means of simple diffusion. Larger molecules (such as DNA or RNA) are mobilized by the electrical current and thus they too can enter the cell. Electroporation is currently used for delivery of ions, drugs, dyes, tracers, antibodies, RNA, and DNA into cells [Gehl, J et al, Acta Physiol Scand, 2003. 177(4): p. 437-47]. The exact translocation mechanism by which electroporated DNA enters the nucleus is not clear, but it seems that the DNA migrates electrophoretically through the membrane and then diffuses toward the nucleus.

Several osteogenic genes are candidates for bone regeneration, particularly those from the TGFβ superfamily. BMPs are known for their ability to induce bone formation in ectopic and orthotopic sites. Recombinant human BMPs are currently used in the clinical setting to create bone, and their application has met with success. Previous reports have shown the feasibility of inducing bone formation by using in vivo electroporation-mediated gene transfer of BMPs [Abdelaal, M. M., et al., J Craniofac Surg, 2004. 15(5): p. 736-41; discussion 742-4; Kawai, M., et al., Hum Gene Ther, 2003. 14(16): p. 1547-56; Kishimoto, K. N., et al., Bone, 2002. 31(2): p. 340-7] in ectopic sites.

### SUMMARY OF THE INVENTION

According to an aspect of some embodiments of the present invention there is provided a method of generating bone or connective tissue in a subject in need thereof, the method comprising:

(a) implanting a scaffold in a damaged region of the subject, the scaffold comprising a tissue growth promoting agent; and

(b) enhancing uptake of the tissue growth promoting agent into cells located on the scaffold, wherein the enhancing is effected by induced poration of the cells thereby generating bone or connective tissue in the subject.

According to an aspect of some embodiments of the present invention there is provided a method of generating bone or connective tissue in a subject in need thereof, the method comprising:

(a) implanting a scaffold in a damaged region of the subject;

(b) administering to the subject a tissue growth promoting agent at a site of the implanting; and

(c) enhancing uptake of the tissue growth promoting agent into cells located on the scaffold, wherein the enhancing is effected by induced poration of the cells, thereby generating bone or connective tissue in the subject.

According to an aspect of some embodiments of the present invention there is provided a kit for generating bone or connective tissue, comprising:

(i) a scaffold;

(ii) a tissue growth promoting agent; and

(iii) instructions for generating the bone or cartilage tissue, the instructions comprising a protocol for enhancing uptake of the tissue growth promoting agent into cells via induced poration.

According to some embodiments of the invention, the induced portation is effected by a method selected from the group consisting of electroporation, sonoporation and laser-induced poration.

According to some embodiments of the invention, the method further comprises seeding the cells on the scaffold prior to the implanting.

According to some embodiments of the invention, the cells comprise stem cells.

According to some embodiments of the invention, the tissue growth promoting agent is selected from the group consisting of a polynucleotide agent, a polypeptide agent, a hormone and a small molecule.

According to some embodiments of the invention, the tissue growth promoting agent comprises an osteogenic agent.

According to some embodiments of the invention, the osteogenic agent is a bone morphogenetic protein (BMP).

According to some embodiments of the invention, the BMP is selected from the group consisting of BMP-2, BMP-4, BMP-6, BMP-7, BMP-9, BMP-12, BMP-13 and BMP-14.

According to some embodiments of the invention, the BMP is BMP-9.

According to some embodiments of the invention, the scaffold does not comprise a viral vector.

According to some embodiments of the invention, the scaffold does not comprise a lipid transfection agent.

According to some embodiments of the invention, the tissue growth promoting agent is not administered as a viral vector.

According to some embodiments of the invention, the tissue growth promoting agent is not administered with a lipid transfection agent.

According to some embodiments of the invention, the scaffold comprises collagen.

According to some embodiments of the invention, the electroporation is effected using needle electrodes.

According to some embodiments of the invention, the enhancing uptake is effected 7 -12 days following the implanting.

According to some embodiments of the invention, the induced poration is effected by a method selected from the group consisting of electroporation, sonoporation and laser-induced poration.

According to some embodiments of the invention, the tissue growth promoting agent is comprised in the scaffold.

According to some embodiments of the invention, the scaffold comprises collagen.

According to some embodiments of the invention, the cells comprise stem cells.

According to some embodiments of the invention, the tissue growth promoting agent comprises an osteogenic agent.

According to some embodiments of the invention, the tissue growth promoting agent is selected from the group consisting of a polynucleotide agent, a polypeptide agent, a hormone and a small molecule.

According to some embodiments of the invention, the osteogenic agent is a bone morphogenetic protein (BMP).

According to some embodiments of the invention, the BMP is selected from the group consisting of BMP-2, BMP-4, BMP-6, BMP-7, BMP-9, BMP-12, BMP-13 and BMP-14.

According to some embodiments of the invention, the BMP is BMP-9.

According to some embodiments of the invention, the kit further comprises a needle electrode for performing the electroporation.

According to some embodiments of the invention, the kit does not comprise a lipid transfection agent.

According to some embodiments of the invention, the tissue growth promoting agent is not comprised in a viral vector.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are photographs illustrating setup of the electroporation system. A 1.5-mm-long defect was made in the mouse radius bone, and a collagen sponge was placed in the defect site. 10 days after surgery plasmids encoding either for Luciferase (pLuc) or BMP-9 (pBMP-9) cDNAs were injected into the defect site, and electroporation was immediately performed using needle electrodes. A) Mouse ready for electroporation. The animal is mounted on a fluoroscope detector and needle electrodes are placed. B) Locations of the electrodes and the needle used for plasmid injection are verified using a fluoroscope.

FIG. 1C is a graph illustrating calibration of the system using pBMP-9 and pBMP-6. X-axis: amount of plasmid injected in vivo; Y-axis: volume of new bone formed (mm³).

FIGS. 2A-C are photographs illustrating that host cells populate the defect site. A 1.5-mm-long defect was made in the mouse radius, and a collagen sponge was placed in the defect site. 10 days after surgery, the radius was harvested and stained using H&E. A-C) Radial defect 10 days after surgery. Staining shows the presence of host progenitor cells (HPCs) (arrowheads). M=defect margin, U=ulna.

FIG. 3 are graphs and photographs illustrating gene transfer efficiency and localization. Bioluminescence imaging (BLI) was used to monitor luciferase activity in bone defects following pLuc injection and electroporation. The x axis displays time post-electroporation; the y axis shows activity in RLUs. *P<0.05, 2-tailed t-test, n=4. Note the representative pictures of one mouse.

FIGS. 4A-H are graphs and photographs illustrating bone formation in the defect area. A) Bone volume analysis: a comparison of newly formed bone in the radial defect in animals in the pBMP-9 with electroporation (BMP9, 6 mice), pLuc with electroporation (Luc, 4 mice), and pBMP-9 without electroporation (No EP, 4 mice) groups and of segments of native radii having the same dimensions (Native, 6 mice). *P<0.01, 2-tailed t-test. B) μCT reconstruction comparison of defects treated with pLuc and electroporation (Luc), defects treated with pBMP-9 and electroporation (BMP9), and defects treated with BMP9 without electroporation (No EP). M=defect margin. Arrowhead indicates new bone formation in the defect. In the 3D images, orange regions denote new bone formation. C-H) Histological sections containing newly formed bone. 1.5-mm-long defects were made in mice radii, and a collagen sponge was placed in the defect site. 10 days post-operation, the radius was injected with either pBMP-9 or pLuc followed by electroporation. As a control, radii were injected with pBMP-9 but electroporation was not performed. 5 weeks post-electroporation, the radii were harvested and stained using Masson's trichome. M=defect margin. U=ulna. Arrowheads indicate new bone formation; asterisks indicate soft tissue in the defect site.

FIGS. 5A-F are bar graphs illustrating quantitative analysis of structural parameters of induced bone formation in the defect area. A-F) Newly formed bone in the radial defect in mice in the pBMP with electroporation group (BMP9) was compared to segments of native radii having the same dimensions (Native). *P<0.05, 2-tailed t-test, n=6 in each group. The structural parameters include: A) trabecular thickness (mm); B) trabecular number (1/mm); C) trabecular separation (mm); D) connectivity density (1/mm³); E) bone volume density (BV/TV, mm/mm); and F) bone mineral density (mg HA/cm³).

FIGS. 6A-F are graphs and photographs illustrating HPC characterization and gene expression. HPCs isolated from defect site 13 days postoperation were assayed to determine their differentiation capabilities, specifically A) osteogenic differentiation (ALP/BCA); B) adipogenic differentiation (Oil red O stain); and C) chondrogenic differentiation (Alcian blue staining of pellet culture) were used. Gene delivery to HPCs was verified in mice 3 days after injection of pLuc in the radial defect followed by electroporation (13 days after creation of the radial defect and implantation of the collagen sponge). D) Results of a BLI study performed in a representative mouse. After they underwent the BLI study, mice were sacrificed and cells from explanted radii were isolated. E) photomicrograph shows HPCs isolated from the radial explants. F) Isolated HPCs were lyzed and mRNA was extracted. Using quantitative RT-PCR using specific primers for the Luc sequence, the presence of Luc expression in the isolated cells was verified (EP Luc). HPCs isolated from defects in which electroporation was not performed were used as a negative control (No EP).

FIGS. 7A-B are images of micro CT scans illustrating new bone formation in radial defect with no collagen sponge implantation after pBMP-9 electroporation using caliper electrodes: When in vivo electroporation of 50 μg pBMP-9 into defect site in which no collagen sponge was implanted, using caliper electrodes (and not needle electrodes), bone formation was not limited to the defect site. Extensive bone formation was evident in adjacent tissues, as well as ectopic bone formation that was not fused to the native bone as all. A, B) μCT reconstruction of defects treated with pBMP-9 and electroporation. Arrowhead indicates new bone formation in the 2D images. In the 3D images, orange regions denote new bone formation.

### DESCRIPTION OF SPECIFIC EMBODIMENTS OF THE INVENTION

The present invention, in some embodiments thereof, relates to methods of generating connective tissue and, more particularly, but not exclusively, to generating bone tissue using a combination of electroporation of tissue growth promoting genes and implantation of scaffolds.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Nonunion fractures present a major challenge to bone repair and, to date, no optimal solution has been identified. The present inventors hypothesized that direct in vivo electroporation to a defect site containing progenitor cells and an osteogenic gene would induce targeted bone regeneration, limited to the fracture site.

Whilst reducing the present invention to practice, the present inventors created a nonunion fracture in the radius of C3H/HeN mice, and implanted a collagen sponge in the defect site. To allow time for recruitment of host progenitor cells (HPCs) into the implanted sponge, the mice were housed for 10 days prior to introduction of the gene. At the time of gene introduction, needle electrodes were inserted into the defect site, guided by fluoroscopy. Immediately thereafter, plasmid DNA encoding for the gene luciferase (pLuc) or BMP-9 (pBMP-9) was injected into the defect site, and electrical pulses were generated by an ECM 830 electroporator. To provide a control group, pBMP-9 was injected into the radial defect of some mice without performing electroporation. Five weeks following electroporation the mice were sacrificed, and their dissected limbs were subjected to micro-computer tomography (μCT) scanning.

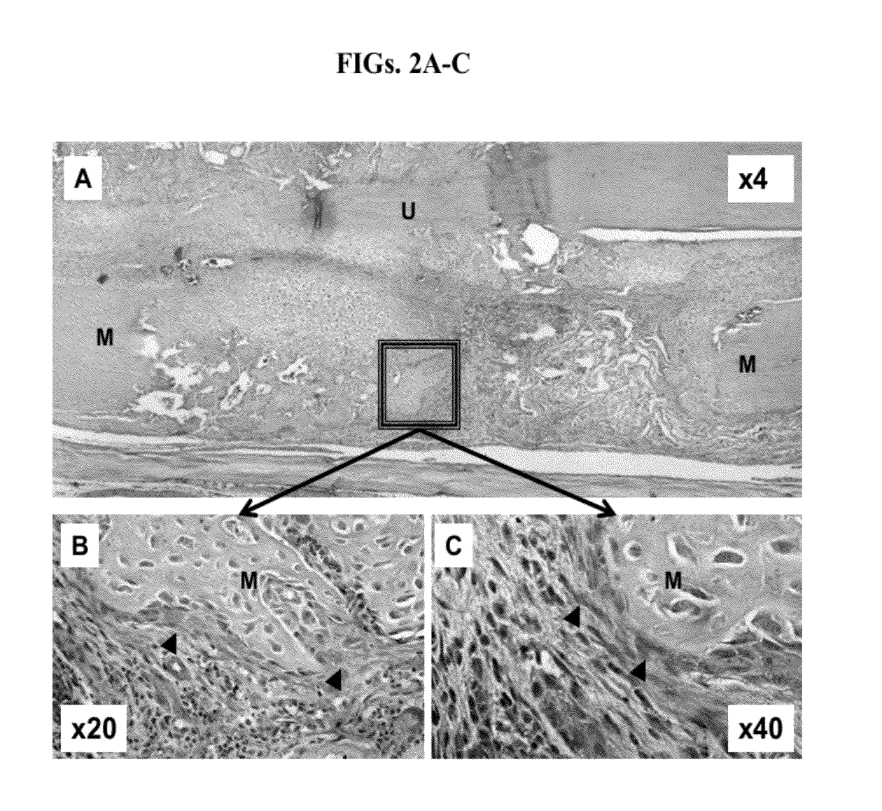

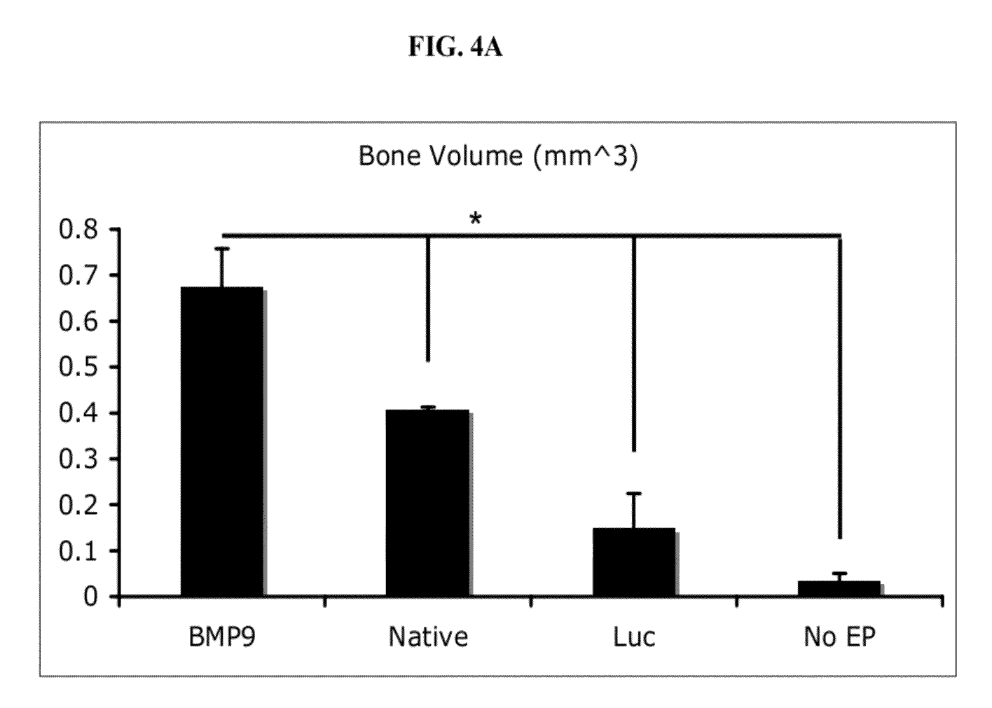

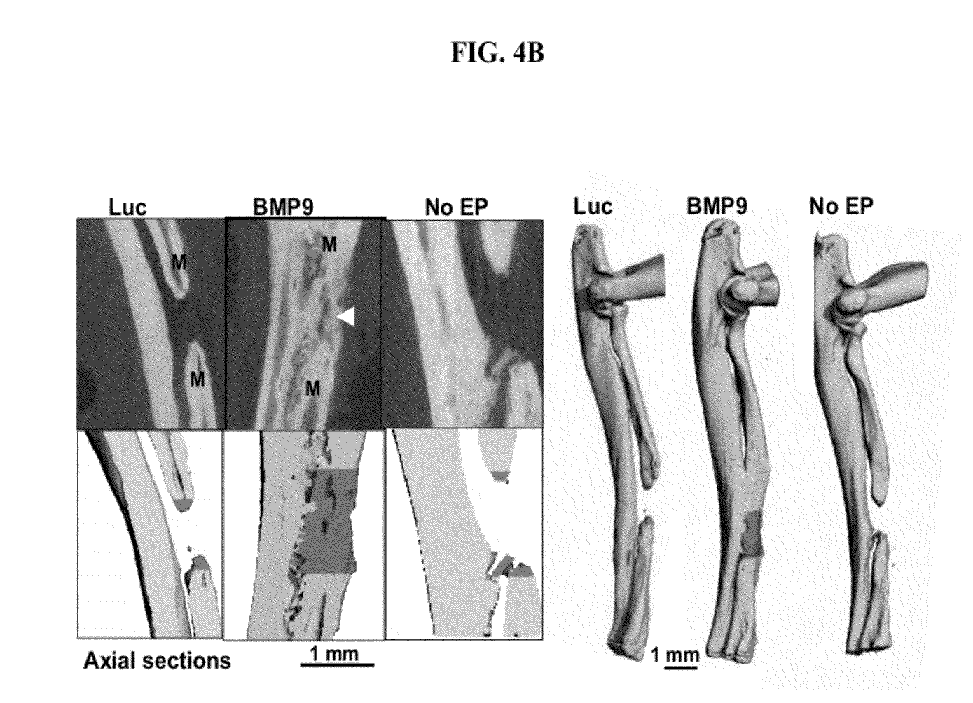

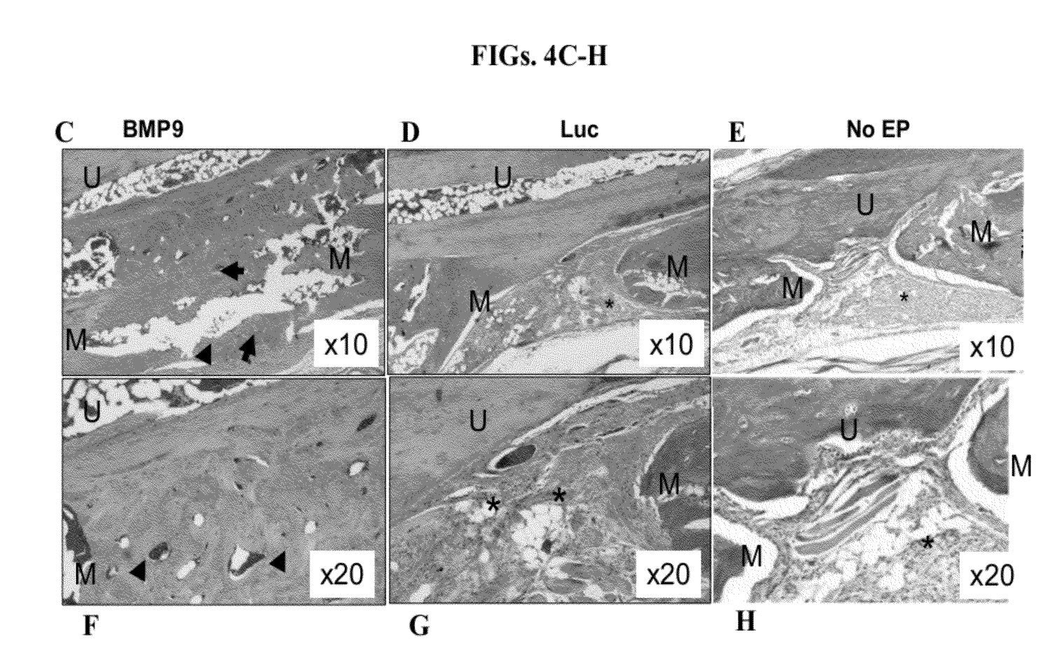

As illustrated in FIGS. 2A-C, host progenitor cells were recruited to the defect site following implantation of the scaffold. The μCT analysis of murine radii that had been electroporated using pBMP9 demonstrated massive bone formation in the site of the bone defect (FIGS. 4A-C). The new bone was fused to the edges of the original defect, fully bridging the bone gap. A small amount of bone growth was also noted at the edges of the defect in mice in which electroporation was performed using pLuc and in mice in which pBMP9 was injected but no electroporation was performed.

The present data indicates, for the first time, that regeneration of bone in a nonunion bone defect can be attained by performing in vivo electroporation with an osteogenic gene combined with recruitment of HPCs. As such, the present inventors postulate that this method may pave the way for regeneration of other connective tissues.

Thus, according to one aspect of the present invention there is provided a method of generating a bone or connective tissue in a subject in need thereof, the method comprising:

(a) implanting a scaffold in a damaged region of the subject, the scaffold comprising a tissue growth promoting agent; and

(b) enhancing uptake of the tissue growth promoting agent into cells located on the scaffold, wherein the enhancing is effected by induced poration of the cells, thereby generating bone or connective tissue in the subject.

As used herein, the phrase “connective tissue” refers to tissues which surround, protect, bind and support all of the structures in the body. Examples of connective tissues include, but are not limited to, cartilage (including, elastic, hyaline, and fibrocartilage), collagen, adipose tissue, reticular connective tissue, embryonic connective tissues (including mesenchymal connective tissue and mucous connective tissue), tendons, ligaments, and bone.

According to the teachings of the present invention various types of bones can be formed and/or repaired using the presently described methods, these include without being limited to, ethmoid, frontal, nasal, occipital, parietal, temporal, mandible, maxilla, zygomatic, cervical vertebra, thoracic vertebra, lumbar vertebra, sacrum, rib, sternum, clavicle, scapula, carpal bones, ilium, ischium, pubis, patella, calcaneus, and tarsal bones. The present invention also contemplates generation of long bones (i.e. bones which are longer than they are wide and grow primarily by elongation of the diaphysis with an epiphysis at the ends of the growing bone). Examples of long bones include femur, tibia, fibula (i.e. leg bones), humerus, radius, ulna (i.e. arm bones), metacarpal, metatarsal (i.e. hand and feet bones), and the phalanges (i.e. bones of the fingers and toes).

As used herein, the term “scaffold” refers to a 3D matrix upon which cells may be cultured (i.e., survive and preferably proliferate for a predetermined time period).

The scaffold can be derived from naturally occurring substances (i.e., protein based) or synthetic substances. Suitable synthetic matrices are described in, e.g., U.S. Pat. Nos. 5,041,138, 5,512,474, and 6,425,222. Materials used to fabricate the scaffolds of the present invention can be natural, synthetic, biocompatible, biodegradable and/or non-biodegradable.

Calcium carbonate, aragonite, and porous ceramics (e.g., dense hydroxyapatite ceramic) are suitable for use in the scaffold. Other contemplated porous materials include, but are not limited to calcium titanate, hydroxylapatite (HA), tricalcium phosphate (TCP) and other calcium phosphates and calcium-phosphorus compounds, hydroxylapatite calcium salts, inorganic bone, dental tooth enamel, aragonite, calcite, nacre, graphite, pyrolytic carbon, bioglass, bioceramic, and mixtures thereof

Alternatively, or additionally, polymers may be used to fabricate the scaffold of the present invention.

According to one embodiment the scaffold material is a hydrogel

According to one embodiment, the polymer with which the scaffold is fabricated is synthetic.

The phrase “synthetic polymer” refers to polymers that are not found in nature, even if the polymers are made from naturally occurring biomaterials. Examples include, but are not limited to, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and combinations thereof.

Suitable synthetic polymers for use according to the teachings of the present invention can also include biosynthetic polymers based on sequences found in collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides and combinations thereof.

According to another embodiment, the polymer from which the scaffold is fabricated is natural.

The phrase “natural polymer” refers to polymers that are naturally occurring. Non-limiting examples of such polymers include, silk, collagen-based materials, chitosan, hyaluronic acid and alginate.

According to one embodiment, the scaffold is comprised of collagen.

Collagen scaffolds are commercially available from Integra Life Sciences Inc. (DuraGen) or Stryker inc. (TissueMend).

The phrase “biocompatible polymer” refers to any polymer (synthetic or natural) which when in contact with cells, tissues or body fluid of an organism does not induce adverse effects such as immunological reactions and/or rejections and the like. It will be appreciated that a biocompatible polymer can also be a biodegradable polymer.

The phrase “biodegradable polymer” refers to a synthetic or natural polymer which can be degraded (i.e., broken down) in the physiological environment such as by proteases. Biodegradability depends on the availability of degradation substrates (i.e., biological materials or portion thereof which are part of the polymer), the presence of biodegrading materials (e.g., microorganisms, enzymes, proteins) and the availability of oxygen (for aerobic organisms, microorganisms or portions thereof), carbon dioxide (for anaerobic organisms, microorganisms or portions thereof) and/or other nutrients. Examples of biodegradable polymers include, but are not limited to, collagen (e.g., Collagen I or IV), fibrin, hyaluronic acid, polylactic acid (PLA), polyglycolic acid (PGA), polycaprolactone (PCL), poly(Lactide-co-Glycolide) (PLGA), polydioxanone (PDO), trimethylene carbonate (TMC), polyethyleneglycol (PEG), Collagen, PEG-DMA, Alginate, chitosan copolymers or mixtures thereof.

The phrase “non-biodegradable polymer” refers to a synthetic or natural polymer which is not degraded (i.e., broken down) in the physiological environment. Examples of non-biodegradable polymers include, but are not limited to, nylon, silicon, silk, polyurethane, polycarbonate, polyacrylonitrile, polyethyleneoxide, polyaniline, polyvinyl carbazole, polyvinyl chloride, polyvinyl fluoride, polyvinyl imidazole, polyvinyl alcohol, polystyrene and poly(vinyl phenol), aliphatic polyesters, polyacrylates, polymethacrylate, acyl-sutostituted cellulose acetates, non-biodegradable polyurethanes, polystyrenes, chlorosulphonated polyolifins, polyethylene oxide, polytetrafluoroethylene, and shape-memory materials such as poly (styrene-block-butadiene), copolymers or mixtures thereof.

It will be appreciated that more than one polymer may be used to fabricate the scaffolds of the present invention. For example, the scaffold may be fabricated from a co-polymer.

The term “co-polymer” as used herein, refers to a polymer of at least two chemically distinct monomers. Non-limiting examples of co-polymers which may be used to fabricate the scaffolds of the present invention include PLA-PEG, PEGT-PBT, PLA-PGA, PEG-PCL and PCL-PLA.

Various methods are known for generating polymeric scaffolds. Exemplary methods include: alignment by surface templating, chemical patterning, nanolithography, electrochemical fabrication, use of a magnetic field, and by shear flow.

According to another embodiment, the scaffold is a hydrogel. The term “hydrogel” as used herein, refers to a class of highly hydrated polymer materials (water content >30% by weight). The hydrogels are composed of hydrophilic polymer chains, which are either synthetic or natural in origin and are commonly used as scaffold in tissue engineering techniques.

A variety of synthetic and naturally derived materials may be used to form hydrogels for tissue engineering purposes. Synthetic materials include poly(ethylene oxide) (PEO), poly(vinyl alcohol) (PVA), poly(acrylic acid) (PAA), poly(propylene furmarate-co-ethylene glycol) (P(PF-co-EG)), and polypeptides. Representative naturally derived polymers include agarose, alginate, chitosan, collagen, fibrin, gelatin, and hyaluronic acid (HA). A subset of these hydrogels (PEO, PVA, P(PF-co-EG), alginate, chitosan, collagen, and HA) are most prevalent use in tissue engineering applications and are preferred in accordance with the present invention.

According to one embodiment, at least one tissue growth promoting agent is incorporated (e.g. attached to, coated on, embedded or impregnated) into the scaffold material or a portion thereof. Such agents can be biological agents such as an amino acid, peptides, polypeptides, proteins, polynucleotide agents e.g. (DNA, RNA, siRNA, oligonucleotides, lipids and/or proteoglycans).

According to one embodiment, the tissue growth promoting agent comprises an osteogenic agent.

As used herein, the phrase “osteogenic agent” refers to an agent that promotes, induces, stimulates, generates, or otherwise effects the production of bone or the repair of bone. The presence of an osteogenic agent in the defect site may elicit an effect on the repair of the defect in terms of shortening the time required to repair the bone, by improving the overall quality of the repair, where such a repair is improved over situations in which such osteogenic agents are omitted, or may achieve contemporaneously both shortened repair times and improved bone quality. It is appreciated that osteogenic agents may effect bone production or repair by exploiting endogenous systems, such as by the inhibition of bone resorption.

Osteogenic agents may promote bone growth by acting as bone anabolic agents. Compositions of the present invention may also effect repair of the bone defect by stabilizing the defect to promote healing. The ramifications of using such osteogenic agents include increased healing rates, effecting a more rapid new bone ingrowth, improved repair quality, or improved overall quality of the resulting bone.

In one embodiment the tissue growth promoting agent is a “small molecule” such as a synthetic molecule, drug, or pharmaceutical involved in, or important to, bone biology, including statins, such as lovastatin, simvastatin, atorvastatin, and the like, fluprostenol, vitamin D, estrogen, a selective estrogen receptor modifier, or a prostaglandin, such as PGE-2. Combinations of such small molecules in providing the osteogenic agent are contemplated herein. In another embodiment the tissue growth promoting agent is a “large molecule” such as an endogenous-derived protein or other protein, an enzyme, a peptide, receptor ligand, a peptide hormone, lipid, or carbohydrate involved in, or important to, bone physiology, including the bone morphogenic or bone morphogenetic proteins (BMPs), such as BMP-2, BMP-4, BMP-6, BMP-7, BMP-9, BMP-12, BMP-13 and BMP-14, chrysalin, osteogenic growth peptide (OGP), bone cell stimulating factor (BCSF), KRX-167, NAP-52, gastric decapeptide, parathyroid hormone (PTH), a fragment of parathyroid hormone, osteopontin, osteocalcin, a fibroblast growth factor (FGF), such as basic fibroblast growth factor (bFGF) and FGF-1, osteoprotegerin ligand (OPGL), platelet-derived growth factor (PDGF), an insulin-like growth factor (IGF), such as IGF-1 and IGF-2, vascular endothelial growth factor (VEGF), transforming growth factor (TGF), such as TGF-alpha and TGF-beta, epidermal growth factor (EGF), growth and differentiation factor (GDF), such as GDF-5, GDF-6, and GDF-7, thyroid-derived chondrocyte stimulation factor (TDCSF), vitronectin, laminin, amelogenin, amelin, fragments of enamel, or dentin extracts, bone sialoprotein, and analogs and derivatives thereof. Combinations of such large molecules in providing the osteogenic agent are contemplated herein.

Additionally and/or alternatively, the scaffolds of the present invention may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin).

As mentioned, the present invention contemplates scaffolds comprising polynucleotide tissue growth promoting agents. Thus, for example, the present invention contemplates nucleic acid constructs (e.g. plasmids) comprising polynucleotides which encode any of the tissue growth promoting polypeptides mentioned herein above.

For instance, a nucleic acid sequence encoding BMP-9 (AF188285.1—SEQ ID NO: 4) or BMP-6 (NM_{—}001718.4—SEQ ID NO: 5) may be ligated into a nucleic acid construct suitable for mammalian cell expression. Such a nucleic acid construct typically includes a promoter sequence for directing transcription of the polynucleotide sequence in the cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the tetracycline-inducible promoter (Zabala M, et al., Cancer Res. 2004, 64(8): 2799-804).

The nucleic acid construct may include additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of the present invention is active in the specific cell population transformed—e.g. stem cells. Examples of contemplated promoters include, but are not limited to CMV, Ubiqitin, PGK and SV40.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase RNA stability (Soreq et al., 1974; J. Mol Biol. 88: 233-45).

Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Nucleic acid constructs containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

According to one embodiment, the nucleic acid construct is not a viral vector.

Immobilization of tissue growth promoting agents to the scaffold can be effected by numerous methods known in the art, including but not limited to soaking the scaffold in a solution of the tissue growth promoting agent followed by a freeze-drying process, or chemically cross-linking the tissue growth promoting agent to the scaffold. According to one embodiment, when the tissue growth promoting agent is a polynucleotide agent, the scaffold does not comprise a lipid transfection agent or the like.

The amount of agent immobilized on a scaffold is calibrated such that the agent acts to induce bone formation and is not toxic to the cell.

As mentioned herein above, following administration of the scaffold comprising the tissue growth promoting agent, induced poration is performed so as to enable uptake of the agent into the cells which have embedded themselves on the scaffold. It will be appreciated that a sufficient time is waited such that the subjects cells (e.g. stem cells) migrate towards the scaffold and embed themselves thereupon prior to the step of enhancing uptake of the tissue growth promoting agent into cells located on the scaffold (e.g. 7-12 days, such as 10 days).

It will be further appreciated that the tissue growth promoting agent may be administered to the subject following implantation of the scaffold and prior to the poration step. According to this embodiment, the tissue growth promoting agent may be administered directly into the site of scaffold implantation (i.e. local administration). Fluoroscopic guidance may be used to inject the tissue growth promoting agent into the correct site. A needle may be used to direct the injected agent into the correct site. The agent is typically administered following migration of cells onto the scaffold (e.g. 7-12 days, for example 10 days). Preferably, the poration step is effected no more than 3 days following administration of the agent, more preferably no more than 2 days, more preferably no more than 1 day, more preferably no more than 12 hours, and even more preferably no more than 1 hour.

According to yet another embodiment, the scaffold is preseeded with cells prior to implantation. In this embodiment, if the scaffold comprises the tissue growth promoting agent, the poration step may be effected immediately following implantation or soon after (e.g. after a few hours or 1 day). If the tissue growth promoting agent is administered following scaffold implantation, it may be administered a few hours, 1 day, 2 days, 5 days or 10 days following implantation. Preferably, the poration step is effected no more than 3 days following administration of the agent, more preferably no more than 2 days, more preferably no more than 1 day, more preferably no more than 12 hours, and even more preferably no more than 1 hour.

As used herein, the term “seeding” refers to plating, placing and/or dropping cells into a scaffold. It will be appreciated that the concentration of cells which are seeded on or within the scaffold depends on the type of cells used and the composition of the scaffold.

Techniques for seeding cells onto or into a scaffold are well known in the art, and include, without being limited to, static seeding, filtration seeding and centrifugation seeding. Static seeding includes incubation of a cell-medium suspension in the presence of the scaffold under static conditions and results in non-uniformity cell distribution (depending on the volume of the cell suspension); filtration seeding results in a more uniform cell distribution; and centrifugation seeding is an efficient and brief seeding method (see for example EP19980203774).

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components.

The cells may comprise a heterogeneous population of cells or alternatively the cells may comprise a homogeneous population of cells. Such cells can be for example, stem cells (such as embryonic stem cells, bone marrow stem cells, cord blood cells, mesenchymal stem cells, adult tissue stem cells), progenitor cells (e.g. progenitor bone cells), or differentiated cells such as chondrocytes, osteoblasts, connective tissue cells (e.g., fibrocytes, fibroblasts and adipose cells), endothelial and epithelial cells. Furthermore, the cells may be of autologous origin or non-autologous origin, such as postpartum-derived cells (as described in U.S. application Ser. Nos. 10/887,012 and 10/887,446). Typically the cells are selected according to the tissue being generated.

According to an embodiment of this aspect of the present invention, the cells are bone or cartilage cells. Non limiting examples of bone and cartilage cells include osteoprogenitor cells, osteoblasts, osteocytes, osteoclasts, and chondrocytes.

As used herein, the phrase “stem cell” refers to cells which are capable of differentiating into other cell types having a particular, specialized function (i.e., “fully differentiated” cells) or remaining in an undifferentiated state hereinafter “pluripotent stem cells”.

It will be appreciated that to support cell growth, the cells are seeded in the scaffold in the presence of a culture medium.

The culture medium used by the present invention can be any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, N.Y., USA).

The culture medium may be supplemented with various antibiotics (e.g., Penicillin and Streptomycin), growth factors or hormones, specific amino acids (e.g., L-glutamin) cytokines and the like.

As mentioned, the present invention contemplates enhancing uptake of the tissue growth promoting agent into the cells that reside in the scaffold using induced poration.

As used herein, the terms “poration” and/or “permeablization” refers to various forms of electrically-medicated poration (electroporation), such as the use of pulsed electric fields (PEFs), nanosecond pulsed electric fields (nsPEFs), ionophoreseis, electrophoresis, electropermeabilization, as well as other energy mediated permeabilization, including x-ray mediated poration, microwave mediated poration, laser-mediated poration, fentosecond laser, sonoporation (mediated by ultrasonic or other acoustic energy), and/or combinations thereof, to create temporary pores in a targeted cell membrane.

Electroporation comprises the application of a pulsed electric field using a pulse voltage device to create transient pores in the cellular membrane and, thereby, an exogenous molecule, e.g., tissue growth promoting agent of the present invention, is delivered to the cell. It is known that adjusting the electrical pulse generated by an electroporation system, nucleic acid molecules can find their way through passageways or pores in the cell that are created during such a procedure (see e.g., U.S. Pat. No. 5,704,908, U.S. Pat. No. 5,704,908, Grossin et al., Joint Bone Spine 70 (2003) 480-482, Abdelaal, M. M., et al., J Craniofac Surg, 2004. 15(5): p. 736-41; discussion 742-4; Kawai, M., et al., Hum Gene Ther, 2003. 14(16): p. 1547-56; Kishimoto, K. N., et al., Bone, 2002. 31(2): p. 340-7, the contents of each being incorporated entirely by reference).

As used herein, “pulse voltage device”, or “pulse voltage injection device” refers to an apparatus that is capable of causing or causes uptake of nucleic acid molecules into the cells of a subject by emitting a localized pulse of electricity to the cells, thereby, causing the cell membrane to destabilize and result in the formation of passageways or pores in the cell membrane. Conventional devices of this type are suitable for use for the delivery of a nucleic acid composition of the present invention. In some embodiments, the device is calibrated to allow one of ordinary skill in the art to select and/or adjust the desired voltage amplitude and/or the duration of pulsed voltage and therefore. A pulse voltage nucleic acid delivery device can include, for example, an electroporation apparatus as described e.g. in U.S. Pat. No. 5,439,440, U.S. Pat. No. 5,704,908, U.S. Pat. No. 5,702,384, PCT No. WO96/12520, PCT No. WO 96/12006, PCT No. WO 95/19805, or PCT No. WO 97/07826. An exemplary apparatus is an ECM 830 electroporator (BTX, Harvard Apparatus, Holliston, Mass., USA).

The pulse voltage device may comprise any sort of electrode such as needle electrodes or caliper electrodes. Preferably needle electrodes are used since these serve to localize the field of electroporation.

According to one embodiment needle electrodes are placed at either side of the bone defect (e.g. 1-2 mm apart) and electrical pulses are delivered directly to the defect site. A guidance mechanism (e.g. fluoroscopic guidance) may be used in order to aid in locating the exact site of insertion. A typical electroporation protocol may be eight 100-V pulses at 20 msec per pulse with a 100-msec interval between pulses.

Sonophoresis (i.e., ultrasound) has been used and described in U.S. Pat. No. 5,656,016 (specifically incorporated herein by reference in its entirety) as a device for enhancing the rate and efficacy of permeation of an agent into a cell—see also Watanuki et al Upsala Journal of Medical Sciences, 2009; 114:242-248; Sheyn et al., Gene Therapy 2007, 1-10, the contents of each being incorporated herein by reference.

The scaffold of the present invention may be comprised in a pack, such as an FDA-approved kit which comprises instructions for promoting generation of a connective tissue, such as bone or cartilage.

A tissue growth promoting agent (e.g. a plasmid encoding a BMP gene) may also be comprised in the pack. The tissue growth promoting agent may be wrapped in an individual packaging inside the kit or the tissue growth promoting agent may be comprised in/on the scaffold.

The kit may also comprise needles which are used as electrodes for electroporation.

The instructions further comprise protocol for inducing poration of cells such as an electroporation protocol or a sonoporation protocol for enhancing uptake of the tissue growth promoting agent into cells. The instructions may also comprise details on how to administer the scaffold and further how to seed cells on the scaffold. The pack may also be accompanied by a notice in a form prescribed by a governmental agency regulating the manufacture, use, or sale of pharmaceuticals, which is reflective of approval by the agency of the form of the compositions for human or veterinary administration. Such notice, for example, may include labeling approved by the U.S. Food and Drug Administration for prescription drugs or of an approved product insert.

Since the scaffolds of the present invention may be used to generate tissue thereon, they may be used for treating diseases characterized by tissue damage or loss (e.g. bone or connective tissue loss).

As used herein, the term “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

As used herein, the phrase “pathology characterized by bone or connective tissue damage or loss” refers to any disorder, disease or condition exhibiting a bone or connective tissue damage (i.e., non-functioning tissue, cancerous or pre-cancerous tissue, broken tissue, fractured tissue, fibrotic tissue, or ischemic tissue) or a bone or connective tissue loss (e.g., following a trauma, an infectious disease, a genetic disease, and the like) which require tissue regeneration. Examples for disorders or conditions requiring bone or connective tissue regeneration include, but are not limited to, bone cancer, articular cartilage defects, musculoskeletal disorders, including degenerative disc disease and muscular dystrophy, osteoarthritis, osteoporosis, osteogenesis, Paget's disease, bone fractures, and the like.

As used herein, the term “subject” refers to mammals, including humans. Preferably, this term encompasses individuals who suffer from pathologies as described hereinabove.

Methods of implanting scaffolds in a subject are known in the art (see for example, Artzi Z, et al., 2005, J. Clin. Periodontol. 32: 193-9; Butler C E and Prieto V G, 2004, Plast. Reconstr. Surg. 114: 464-73).

Preferably the scaffold is implanted at a ligament, tendon, cartilage, intervertebral disc or bone tissue.

Thus for example, when administration of the scaffold is for bone regeneration, the scaffold is placed at a desired location in bone in such conditions such as non-union fractures, osteoporosis, of periodontal disease or defect, osteolytic bone disease, post-plastic surgery, post-orthopedic implantation, post neurosurgical surgery that involves calvaria bone removal, in alveolar bone augmentation procedures, for spine fusion and in vertebral fractures.

When the administration of the scaffold is for generation of tendon/ligament tissue, the scaffold is placed at a desired location in tendon/ligament following tissue tear due to trauma or inflammatory conditions.

When the administration of the scaffold is for generation of cartilage tissue, the scaffold is placed at a desired location in cartilage to treat defects due to Rheumatoid Arthritis, Osteoarthritis, trauma, cancer surgery or for cosmetic surgery.

When the administration of the scaffold is for generation of intervertebral disc tissues including nucleous pulposus and annulus fibrosus, the scaffold is placed at a desired location of nucleous pulposus degeneration, annulus fibrosus tears, or following nucleotomy or discectomy.

Placing the scaffold in the desired location may be done by direct administration such as by injection, or by implantation of a solid tissue graft.

As mentioned, the cells which can be used according to the teachings of the present invention may comprise non-autologous cells.

Non-autologous cells (e.g. allogeneic cells or xenogeneic cells), such as human cadavers, human donors or xenogeneic donors (e.g. porcine), may induce an immune reaction when administered to the subject. Several approaches have been developed to reduce the likelihood of rejection of non-autologous cells. These include either suppressing the recipient immune system or encapsulating the non-autologous cells in immunoisolated, semipermeable membranes before transplantation.

Encapsulation techniques are generally classified as microencapsulation, involving small spherical vehicles and macroencapsulation, involving larger flat-sheet and hollow-fiber membranes (see for example, Uludag, H. et al. Technology of mammalian cell encapsulation. Adv Drug Deliv Rev. 2000; 42: 29-64). Pollok et al were able to successfully encapsulate a polymer scaffold seeded with islets using porcine chondrocytes [Dig Surg 2001; 18:204-210].

Methods of preparing microcapsules are known in the arts and include, for example, those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules may be prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

Examples of immunosuppressive agents which can be used to minimize immunosuppression include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF.alpha. blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

The scaffold and/or tissue growth promoting agent of the present invention may be implanted/administered to a subject per se, or it may be mixed with suitable carriers or excipients.

Hereinafter, the term “carrier” refers to a carrier or a diluent that does not cause significant irritation to an organism and does not abrogate the biological activity and properties of the scaffold. Exemplary carriers include Hank's solution, Ringer's solution, or physiological salt buffer.

Typically a therapeutically effective amount of tissue growth promoting agent is one which is effective to prevent, alleviate or ameliorate symptoms of a disorder (e.g., bone or connective tissue disorder) or generate a therapeutic amount of tissue in the subject being treated.

Determination of a therapeutically effective amount is well within the capability of those skilled in the art, especially in light of the detailed disclosure provided herein.

For any preparation used in the methods of the invention, the therapeutically effective amount or dose can be estimated from animal models to achieve a desired concentration or titer. Such information can be used to more accurately determine useful doses in humans.

Toxicity and therapeutic efficacy of the active ingredients described herein can be determined by standard pharmaceutical procedures in experimental animals. The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide cell numbers sufficient to induce tissue regeneration (e.g. bone and cartilage formation). The minimal effective concentration (MEC) will vary for each preparation, but can be estimated from in vitro data. Dosages necessary to achieve the MEC will depend on individual characteristics and route of administration. Detection assays can be used to determine plasma concentrations.

The amount of scaffold and tissue growth promoting agent to be administered will, of course, be dependent on the subject being treated, the severity of the affliction, the manner of administration, the judgment of the prescribing physician, etc.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes Cellis, J. E., ed. (1994); “Culture of Animal Cells - A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Plasmids: Plasmids encoding for the genes luciferase (SEQ ID NO: 6; pLuc) and BMP-9 (SEQ ID NO: 4; pBMP-9) were amplified using standard procedures and purified using an EndoFree Kit (Qiagen, Valancia, Calif., USA).

Surgical procedure: All animals were provided with water and food ad libitum throughout the duration of the study. In all in vivo experiments mice (C3H/Hen females, 8-10 weeks old) were anesthetized with a mixture of xylazine and ketamine (0.15% xylazine and 0.85% ketamine), which was injected intraperitoneally (IP) at 1 μl/g body weight. The method by which a nonunion segmental radial fracture was created was based on a previously described protocol Moutsatsos, I. K., et al., Mol Ther, 2001. 3(4): p. 449-61, the contents of which are incorporated herein by reference. Briefly, female C3H/HeN mice, each between 8 and 10 weeks of age, were anesthetized in the manner described above. The skin was cut and a 1.5-mm-long defect was created in the right radius bone. A 1.5-mm-long collagen sponge (DuraGen, Integra Neurosciences, Plainsboro, N.J., USA) was implanted into the defect and the skin was sutured. Surgery was performed in 27 mice, and the animals were randomly allocated to one of the following groups: pBMP-9 with electroporation (n=6), pBMP-9 without electroporation (n=4), pLuc with electroporation (n=4), histological evaluation of cells populating the defect (n=3), host progenitor cell (HPCs) isolation of HPCs after electroporation (n=5), and HPCs isolation of HPCs from defects in which electroporation was not performed (n=5).

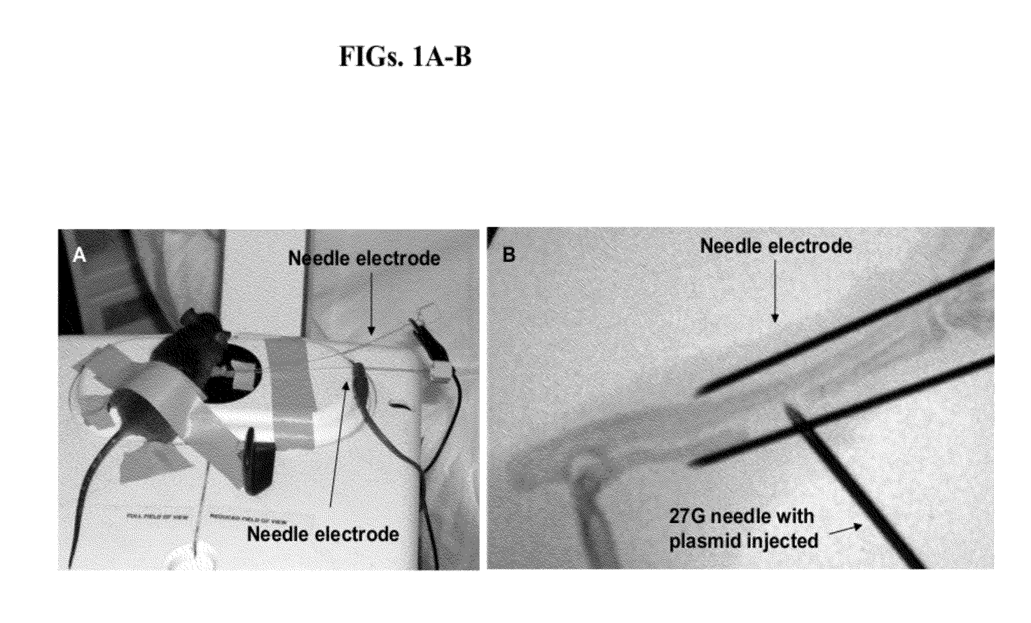

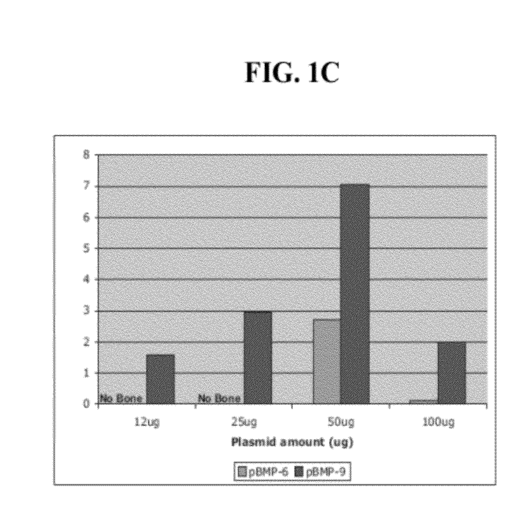

Electroporation: Regeneration of bone in the radial defect was induced by pBMP9 injection followed by in vivo electroporation 10 days after the operation. A mixture of pBMP-9 in phosphate-buffered saline (50 μg pBMP-9 in 18 μl PBS×1) was injected into the radial defect site in 6 mice by using a 27-gauge needle under fluoroscopic guidance. Immediately after the injection, in vivo electroporation was performed. Using needle electrodes, which were placed at both sides of the radial defect (1-2 mm apart) under fluoroscopic guidance, electrical pulses were delivered directly to the defect site (FIGS. 1A-B). To generate these electrical pulses, an ECM 830 electroporator was used (BTX, Harvard Apparatus, Holliston, Mass., USA). For each procedure 8 100-V pulses at 20 msec per pulse with a 100-msec interval between pulses was used.

In order to determine the amount and type of BMP plasmid to be used, the osteogenic efficiency of two genes were tested: BMP-6 and BMP-9. Twelve, 25, 50 or 100 μg of pBMP-9 or pBMP-6 were injected into the right thigh muscle, and electroporation was performed in vivo. As a control, pLuc or PBSx1 were injected to the left thigh muscle. Bone formation was analyzed using micro CT (Desktop μCT 40, Scanco Medical AG, Bassersdorf, Switzerland)—FIG. 1C.

To monitor gene expression in the defect site (and for use as a control), the same procedure as described above was performed in 4 animals using 50 μg pLuc in 18 μl PBS×1. In a different control group, 50 μg pBMP-9 was injected into radial defects in 4 mice 10 days post-operation and no electroporation was performed. All radii were harvested 5 weeks after electroporation and/or plasmid injection, and analyzed for bone formation by performing μCT and histological studies.

Histological analysis of scaffold population by recipient cells: To evaluate the population of the scaffold implanted in the defect site by HPCs that received gene therapy via electroporation (recipient cells), 3 mice were sacrificed at 5, 7, or 10 days post-operation. Tissue samples were removed and then processed and stained for histological analysis. Briefly, tissue samples were fixed in 70% ethanol, passed through a graded series of ethanol solutions, and embedded in paraffin. Sections (5 μm thick) were cut from each paraffin block by using a motorized microtome (Leica Microsystems GmbH, Wetzlar, Germany). Hematoxylin and eosin (H&E) staining was performed to evaluate the morphological characteristics of the tissue. The analysis showed that on Day 10 post-operation, the implanted collagen sponge was populated by HPCs (FIGS. 2A-C), and thus this time point was chosen for gene delivery.

Imaging of luciferase expression in the fracture site: Luciferase expression in the defect site was quantified using a bioluminescence imaging (BLI) system. Briefly, this cooled charge-coupled device (CCCD) tracking system consists of a CCCD camera (model LN/CCD-1300EB) equipped with an ST-133 controller and a 50-mm Nikon lens (Roper Chemiluminescence Imaging System, Roper Scientific, Princeton Instrument, Trenton, N.J., USA). In this system, a pseudocolor image represents light intensity (blue signifies least intense and red most intense). The integrated light is the result of a 2-min exposure and acquisition. Before light detection, the mice were anesthetized with a ketamine-xylazine mixture. Ten minutes prior to monitoring of light emission, the animals were given IP injections of beetle luciferin (Promega, Madison, Wis., USA) in PBS (126 mg/kg body weight).The mice were then exposed to the CCCD system and the composite image was transferred to a personal computer by using a plug-in module for further analysis.

Immunohistochemical analysis of luciferase at the fracture site: An immunohistochemical analysis was performed using the Histomouse™-Broad Spectrum kit (Zymed Laboratories, South San Francisco, Calif., USA). In brief, sections of tissue were deparaffinized by soaking them in xylene. The sections were subsequently hydrated in baths of a descending series of ethanol solutions and rinsed in PBS. Endogenous peroxidase activity was removed by treatment with 3% H₂O₂ for 10 minutes. Primary polyclonal anti-luciferase antibody (Cortex Biochem, San Leandro, Calif., USA), diluted 1:100 in PBS, was applied to the slides for 1 hour at room temperature. After incubation with the primary antibody, the slides were rinsed in PBS and a secondary antibody was applied to the slides at room temperature for 10 minutes. After they were washed with PBS (2-5 minutes), the slides were incubated with horseradish peroxidase conjugated to streptavidin and then stained with 3-amino-9-ethyl-carbazole dye for visualization with light microscopy. The slides were stained with hematoxylin, washed, and mounted.

Analysis of bone formation and structure: Mice were sacrificed 5 weeks after gene delivery; their front right limbs were harvested, fixed in 10% formalin, and scanned using a high-resolution microcomputer tomography (μCT) system (Desktop CT 40, Scanco Medical AG, Bassersdorf, Switzerland). Microtomographic slices were acquired at 1000 projections and reconstructed at a spatial nominal resolution of 12 μm. A constrained three-dimensional (3D) Gaussian filter (σ=0.8 and support=1) was used to partly suppress the noise in the volumes. The mineralized tissue was segmented using a global thresholding procedure [Steinhardt, Y., et al., Maxillofacial-derived stem cells regenerate critical mandibular bone defect. Tissue Eng Part A, 2008. 14(11): p. 1763-73]. In addition to the visual assessment of structural images, morphometric indices were determined from the microtomographic datasets by using direct 3D morphometry. Structural metrics measured using μCT are closely correlated with those measured using standard histomorphometry. The following morphometric indices were determined: (i) total volume of bone tissue, including new bone and cavities (TV, mm³); (ii) volume of bone tissue (BV, mm³); (iii) bone tissue density, the BV/TV ratio; (iv) bone mineral density; (v) trabecular thickness (mm); (vi) trabecular number (1/mm); (vii) trabecular separation (mm); and (viii) connectivity density (1/mm³). All structural parameters of the newly formed bone were compared with untreated contralateral radii.

Isolation and differentiation of HPCs from the fracture site: 13 days post-operation, collagen sponges that had been implanted in the defect site were retrieved and washed thoroughly with sterile PBS. Explants were minced into small pieces using sterile tools and digested in a collagenase solution (3 mg/ml collagenase D, Roche Diagnostics GmbH, Mannheim, Germany) in Dulbecco's Modified Eagle Medium (DMEM) at 37° C. The digestion solution was centrifuged (5 min, 4° C., 1200 rpm), and the digested cells were plated in a culture dish in complete DMEM containing 20% fetal bovine serum (FBS). Cells were incubated for 24 hours, washed with PBS, and cultured in medium containing 10% FBS until the cells reached confluence (14 days). The cells were then assayed for their ability to differentiate down osteogenic, adipogenic, and chondrogenic lineages by using the alkaline phosphatase (ALP) assay (normalized to total protein content, using BCA assay), Oil red O stain, and Alcian blue stain, respectively.

Transgene expression in isolated HPCs: To verify transgene expression in HPCs that had been isolated from the defect site, an additional group of 5 mice underwent surgery as described earlier. Ten days after the radial defect had been created, 25 μg pLuc was injected into the defect site and electroporation was performed. Three days later, gene activity was imaged using the BLI system and found to be localized to the defect site. The mice were then sacrificed, and cells were isolated from retrieved scaffolds, as noted above. Total RNA was purified from the isolated cells 24 hours after cell isolation by using TRIzol reagent (Invitrogen Life Technologies, Paisley, UK) according to the manufacturer protocol. Reverse transcription—polymerase chain reaction (RT-PCR) was performed using 2 μg total RNA. Real-time PCR was performed for the Luc gene by using an ABI 7400 Real Time PCR system (Applied Biosystems Foster City, Calif., USA) according to the manufacturer's protocol. The following primers were used: Luc forward primer: GACGAACACTTCTTCATCGTTGAC (SEQ ID NO: 1); reverse primer: GGGTGTTGGAG CAAGATGGA (SEQ ID NO: 2); TaqMan probe: fam-CTGAAGTCTCGATTAAGTAC-bq (SEQ ID NO: 3).

Statistical analysis: A 2-tailed Student t-test was performed to determine significant differences between experimental and control groups. The p value was set at a value of less than 0.05. Results are presented as means ±standard errors.

### Example 1

### Population of the Implanted Collagen Sponge by HPCs

A histological analysis of harvested radii performed 10 days after the operation revealed that the implanted collagen sponge was populated with HPCs (FIGS. 2A-C), forming dense cellular tissue. In areas close to the defect edges cartilage and callus formation was identified, and in the center of the defect dense connective tissue was found. Radii analyzed 5 or 7 days after generation of the defect contained fewer HPCs and more inflammatory cells (not shown).

### Example 2

### Gene Transfer Efficiency and Localization

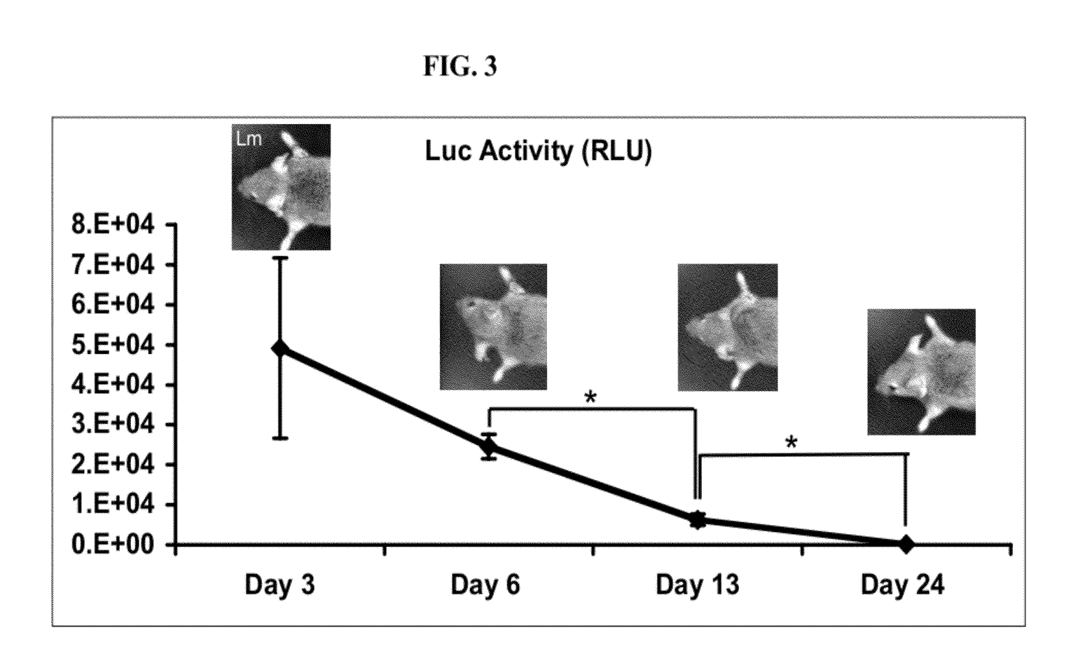

Luciferase activity in the defect site following injection of pLuc and in vivo electroporation was measured using the BLI system. A decrease in the luciferase signal was noted between Day 3 and Day 24 post-electroporation, when no activity was noted. On Day 3 post-electroporation, luciferase activity was 49,126±22,549 RLU, whereas on Day 6 post-electroporation luciferase activity had reduced almost 50% to 24,556±2,999 RLU. This trend in diminished activity continued: luciferase activity measured only 6,268±1,417 RLU on Day 13 and no activity could be detected on Day 24 (FIG. 3). Luciferase activity was significantly reduced between Days 6 and 13 and again between Days 13 and 24 (p<0.05, 2-tailed t-test, n=4). Luciferase activity was localized to the defect site, where it displayed transient activity, as expected when a nonviral gene delivery method is used.

### Example 3

### Bone Formation at the Fracture Site

New bone that had formed in radial defects after injection of pBMP-9 or pLuc and electroporation was compared with new bone in a gene-delivery control group (pBMP-9 injection without electroporation) and with native radial bone of the same dimensions by performing μCT analysis. Significantly more bone formed in the defect site in animals that received pBMP-9 with electroporation than in animals that received pLuc with electroporation, BMP-9 injection without electroporation, or no defect or treatment (intact bone controls). Using a μCT quantitative analysis, 0.6731±0.08 mm³ new bone was measured in the pBMP-9 with electroporation group. Smaller amounts of new bone were found in the pLuc with electroporation group and in the pBMP-9 without electroporation group: 0.14±0.02 mm³ and 0.03±0.02 mm³, respectively (FIG. 4A, *P<0.01, 2-tailed t-test. n=6 for the pBMP-9 with electroporation group and the native bone group; n=4 for the pLuc with electroporation group and the pBMP-9 without electroporation group).

Almost no bone formed in the defect when pLuc was used with electroporation. However, pBMP-9 with electroporation induced rapid bone formation that was localized to the defect site. When pBMP-9 was injected into the defect site but no electroporation was applied, virtually no bone formed—similar to the effect of nonosteogenic gene delivery. FIG. 4B displays μCT images of representative samples from three groups: pBMP-9 with electroporation, pLuc with electroporation, and pBMP-9 without electroporation.

Similar to the μCT data, bone formation and full regeneration of the bone defect was noted only in samples that had been electroporated using pBMP-9. In samples that had been either electroporated using pLuc or injected with pBMP-9 but not electroporated, no bone formed and connective tissue was evident in the defect site. FIGS. 4C-H depicts histological sections from representative samples from each of the experimental groups, which were stained with Masson's trichrome.

### Example 4

### Structural Analysis of Bone Formed at the Fracture Site

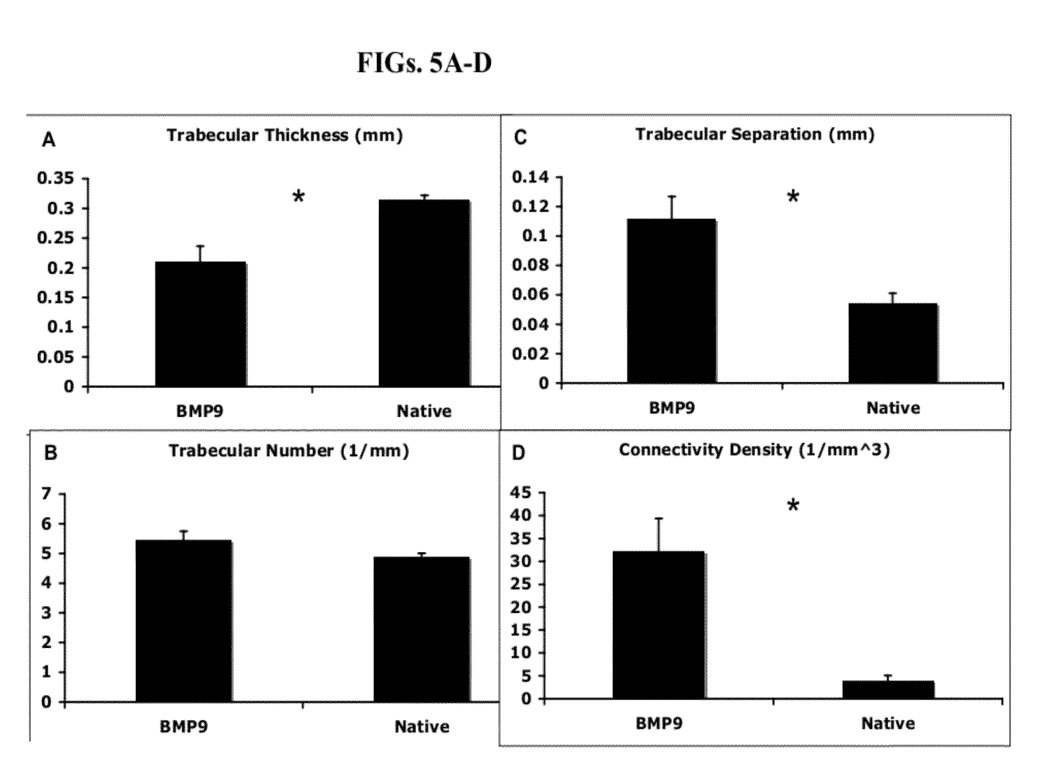

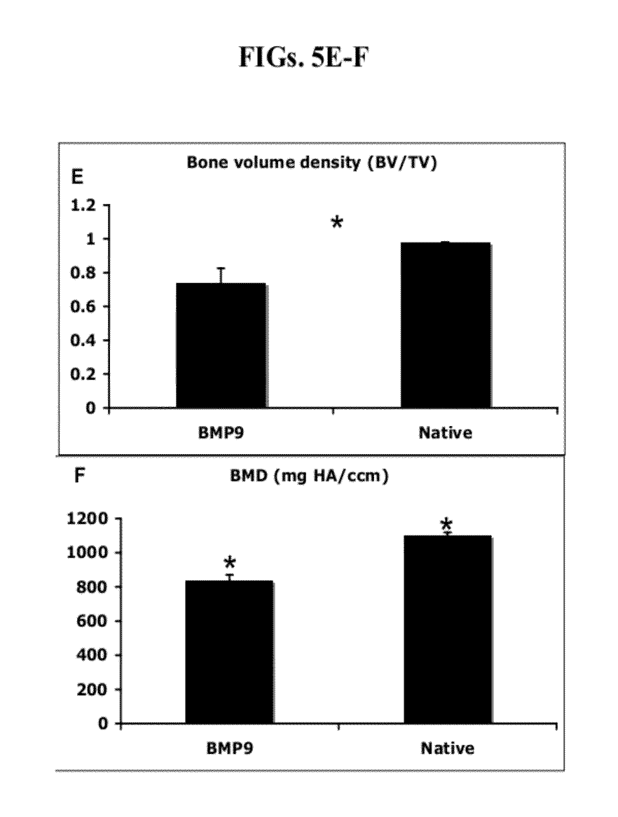

When structural parameters of the new bone were compared with those of native radii, all parameters, except for trabecular number, differed in a statistically significantly manner, although new bone values approached those of the native bone (FIGS. 5A-F). The trabecular thickness of the newly formed bone (0.20±0.02 mm) was significantly lower than that of native bone (0.31±0.008 mm). Both trabecular separation and connectivity density were significantly greater in newly formed bone (0.11±0.01 mm and 32.08±7.29/mm³, respectively) than in native bone (0.05±0.007 mm and 3.78±1.26/mm³, respectively). There was no significant difference in trabecular number: the values were 5.45±0.13/mm³ for new bone and 4.86±0.13/mm³ for native bone. Bone volume density and bone mineral density (BMD) in new bone (0.73±0.08 mm/mm and 836±34 mg HA/cm³, respectively) were significantly lower than those in native bone (0.977±0.002 mm/mm and 1100±18 mg HA/cm³, respectively).

### Example 5

### Characterization of HPCs Isolated from the Defect Site

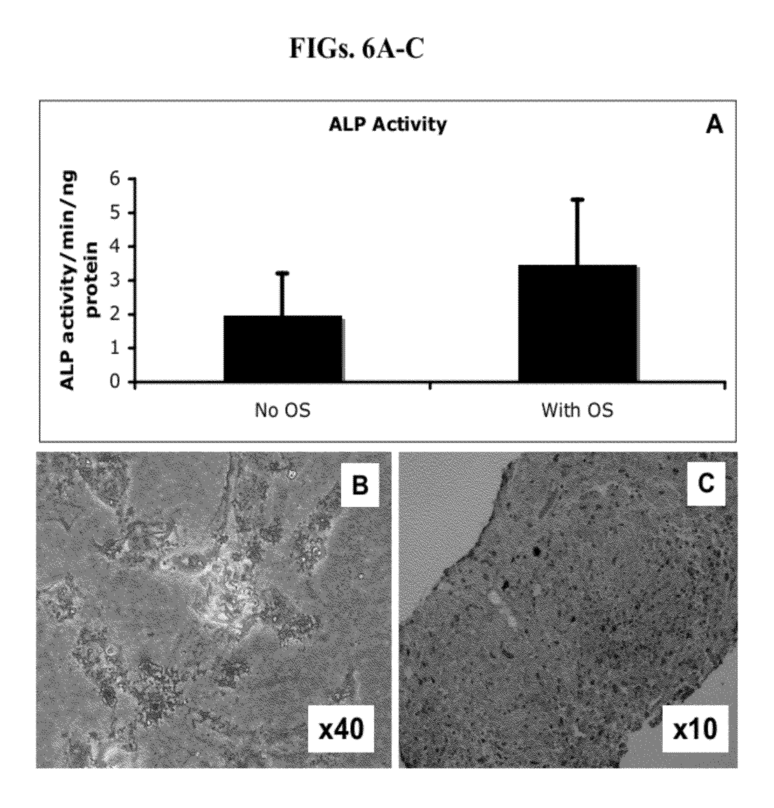

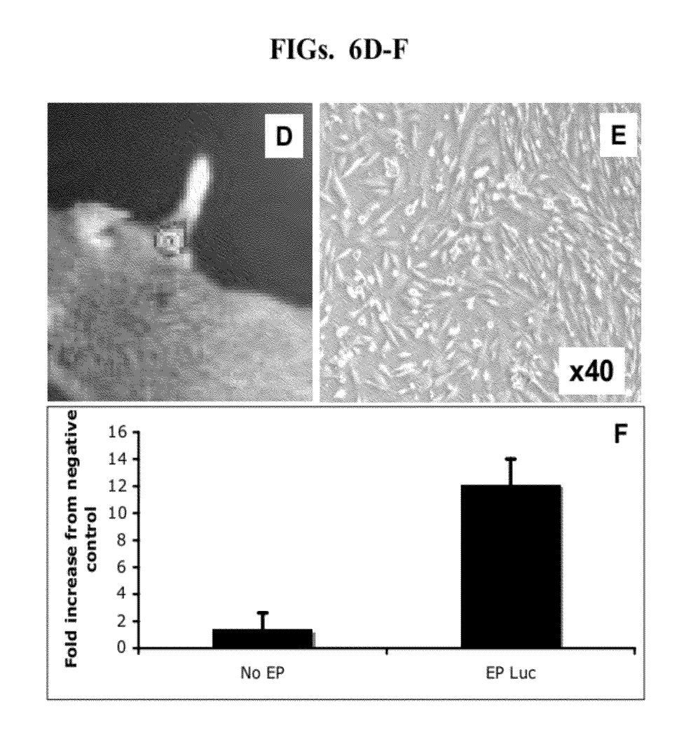

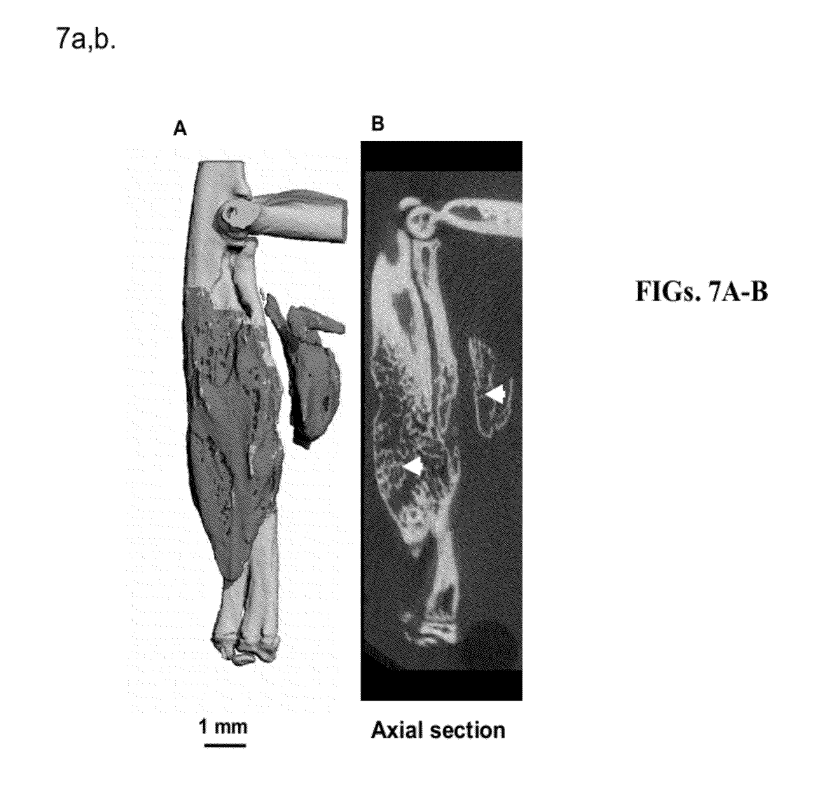

HPCs from the defect site were isolated 13 days after generation of the radial defect (3 days after gene delivery using electroporation) by plastic adherence technique, and the cells were subjected to differentiation assays. Data obtained from the ALP assay and from Oil red O and Alcian blue staining showed the ability of these cells to differentiate along the osteogenic, adipogenic, and chondrogenic lineages, respectively (FIG. 6A-C).

In a different experiment performed to verify transgene expression in isolated HPCs, radial bone defects were created, as previously described, and 10 days later 25 pLuc was injected and electroporation performed in the defect site. Three days following this procedure, gene activity was imaged using the BLI system (FIG. 6D) and found to be localized to the defect site alone. The mice were sacrificed and cells from the defect site were isolated using the plastic adherence technique (FIG. 6E). RNA was purified from the cells 24 hours after their isolation, and RT-PCR following by real-time PCR analysis was performed to verify gene expression in the isolated HPCs. Although no luciferase expression was found in HPCs isolated from radii that were not electroporated, a robust gene expression was found in cells isolated after pLuc injection and in vivo electroporation (FIG. 6E)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of generating bone or connective tissue in a subject in need thereof, the method comprising:
(a) implanting a scaffold in a damaged region of the subject, said scaffold comprising a tissue growth promoting agent; and
(b) enhancing uptake of said tissue growth promoting agent into cells located on said scaffold, wherein said enhancing is effected by induced poration of said cells thereby generating bone or connective tissue in the subject.

2. A method of generating bone or connective tissue in a subject in need thereof, the method comprising:
(a) implanting a scaffold in a damaged region of the subject;
(b) administering to the subject a tissue growth promoting agent at a site of said implanting; and
(c) enhancing uptake of said tissue growth promoting agent into cells located on said scaffold, wherein said enhancing is effected by induced poration of said cells, thereby generating bone or connective tissue in the subject.

3. The method of claim 1, wherein said induced portation is effected by a method selected from the group consisting of electroporation, sonoporation and laser-induced poration.

4. The method of claim 1, further comprising seeding said cells on said scaffold prior to said implanting.

5. The method of claim 1, wherein said cells comprise stem cells.

6. The method of claim 1, wherein said tissue growth promoting agent is selected from the group consisting of a polynucleotide agent, a polypeptide agent, a hormone and a small molecule.

7. The method of claims 1, wherein said tissue growth promoting agent comprises an osteogenic agent.

8. The method of claim 7, wherein said osteogenic agent is a bone morphogenetic protein (BMP).

9-14. (canceled)

15. The method of claim 1, wherein said scaffold comprises collagen.

16. The method of claim 1, wherein said induced poration is effected using needle electrodes.

17. The method of claim 1, wherein said enhancing uptake is effected 7-12 days following said implanting.

18. A kit for generating bone or connective tissue, comprising:
(i) a scaffold;
(ii) a tissue growth promoting agent; and
(iii) instructions for generating the bone or cartilage tissue, the instructions comprising a protocol for enhancing uptake of said tissue growth promoting agent into cells via induced poration.

19. (canceled)

20. The kit of claim 18, wherein said tissue growth promoting agent is comprised in said scaffold.

21. The kit of claim 18, wherein said scaffold comprises collagen.

22. (canceled)

23. The kit of claim 18, wherein said tissue growth promoting agent comprises an osteogenic agent.

24. (canceled)

25. The kit of claim 23, wherein said osteogenic agent is a bone morphogenetic protein (BMP).

26-27. (canceled)

28. The kit of claim 18, further comprising a needle electrode for performing said induced poration.

29-30. (canceled)

31. The method of claim 2, further comprising seeding said cells on said scaffold prior to said implanting.

32. The method of claim 2, wherein said cells comprise stem cells.

33. The method of claim 2, wherein said tissue growth promoting agent is selected from the group consisting of a polynucleotide agent, a polypeptide agent, a hormone and a small molecule.

34. The method of claim 2, wherein said tissue growth promoting agent comprises an osteogenic agent.

35. The method of claim 34, wherein said osteogenic agent is a bone morphogenetic protein (BMP).

36. The method of claim 2, wherein said scaffold comprises collagen.

37. The method of claim 2, wherein said induced poration is effected using needle electrodes.

38. The method of claim 2, wherein said enhancing uptake is effected 7-12 days following said implanting.


SEQUENCE LISTING (6 sequences)

SEQ ID NO: 1 - DNA, 24 bp, Artificial sequence
  Single strand DNA oligonucleotide
gacgaacacttcttcatcgttgac

SEQ ID NO: 2 - DNA, 20 bp, Artificial sequence
  Single strand DNA oligonucleotide
gggtgttggagcaagatgga

SEQ ID NO: 3 - DNA, 21 bp, Artificial sequence
  Single strand DNA oligonucleotide
ctgaagtctctgattaagtac

SEQ ID NO: 4 - DNA, 1942 bp, Homo sapiens
cggtccagcccggcagcgggtgagagtgggtgctggccaggacggttccttcagagcaaacagcagggagatgccggcccgctccttcccagctcctccccgtgcccgctaacacagcacggccgcctgcagtctcctctctgggtgattgcgcgggcctaagatgtgtcctggggcactgtgggtggccctgcccctgctgtccctgctggctggctccctacaggggaagccactgcagagctggggacgagggtctgctgggggaaacgcccacagcccactgggggtgcctggaggtgggctgcctgagcacaccttcaacctgaagatgtttctggagaacgtgaaggtggatttcctgcgcagccttaacctgagtggggtcccttcgcaggacaaaaccagggtggagccgccgcagtacatgattgacctgtacaacaggtacacgtccgataagtcgactacgccagcgtccaacattgtgcggagcttcagcatggaagatgccatctccataactgccacagaggacttccccttccagaagcacatcttgctcttcaacatctccattcctaggcatgagcagatcaccagagctgagctccgactctatgtctcctgtcaaaatcacgtggacccctctcatgacctgaaaggaagcgtggtcatttatgatgttctggatggaacagatgcctgggatagtgctacagagaccaagaccttcctggtgtcccaggacattcaggatgagggctgggagaccttggaagtgtccagcgccgtgaagcgctgggtccggtccgactccaccaagagcaaaaataagctggaagtgactgtggagagccacaggaagggctgcgacacgctggacatcagtgtccccccaggttccagaaacctgcccttctttgttgtcttctccaatgaccacagcagtgggaccaaggagaccaggctggagctgagggagatgatcagccatgaacaagagagcgtgctcaagaagctgtccaaggacggctccacagaggcaggtgagagcagtcacgaggaggacacggatggccacgtggctgcggggtcgactttagccaggcggaaaaggagcgccggggctggcagccactgtcaaaagacctccctgcgggtaaacttcgaggacatcggctgggacagctggatcattgcacccaaggagtatgaagcctacgagtgtaagggcggctgcttcttccccttggctgacgatgtgacgccgacgaaacacgctatcgtgcagaccctggtgcatctcaagttccccacaaaggtgggcaaggcctgctgtgtgcccaccaaactgagccccatctccgtcctctacaaggatgacatgggggtgcccaccctcaagtaccattacgagggcatgagcgtggcagagtgtgggtgcaggtagtatctgcctgcggggctggggaggcaggccaaaggggctccacatgagaggtcctgcatgcccctgggcacaacaaggactgattcaatctgcatgccagcctggaggaggaaagggagcctgctctccctccccacaccccacccaaagcatacaccgctgagctcaactgccagggaaggctaaggaaatggggatttgagcacaacaggaaagcctgggagggttgttgggatgcaaggaggtgatgaaaaggagacagggggaaaaataatccatagtcagcagaaaacaacagcagtgagccagaggagcacaggcgggcaggtcactgcagagactgatggaagttagagaggtggaggaggccagctcgctccaaaacccttggggagtagagggaaggagcaggccgcgtgtcacacccatcattgtatgttatttcccacaacccagttggaggggcatggcttccaatttagagacccg

SEQ ID NO: 5 - DNA, 1542 bp, Homo sapiens
atgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaa

SEQ ID NO: 6 - DNA, 2063 bp, Artificial sequence
  A plasmid encoding the Luciferase gene (pLuc)
atcttttttaccatcgagtataataattttctttataataggtaaacgtgattgtactcctagaattttctgcaatgagtttctagatgtaaatactacagttggctgtgagatactcaagctgttatacaattcacgttcgttataaatatcgttagttaccgtcacagaagtaaactagtaaagccaccatgtcaatagagaataacattttgataggaccacctccgtactatcctttggaagaaggtactgcgggagaacaattacacagagccatatcacgatatgccgcagttccaggaacactagcttatacagatgtacacaccgaacttgaagttacttataaggagtttttagatgtaacatgtcgcttagctgaagctatgaagaactatggcttaggcttacagcatactatttctgtatgtagtgaaaactgcgtacaattctttatgccaatttgcgctgctttatatgttggggttgcaaccgcgcctacaaacgatatttataacgaacgtgaattgtataacagcttgagtatctcacagccaactgtagtatttacatctagaaactcattgcagaaaattctaggagtacaatcacgtttacctattataaagaaaattattatactcgatggtaaaaaagattacttggggtatcaatctatgcagtccttcatgaaagaacacgttcctgccaatttcaatgtatcagcatttaaaccactttcatttgatcttgaccgagttgcatgtattatgaactcttcaggttctacgggattaccaaaaggtgtaccaatatcgcacagaaacaccatatacaggttttcccattgcagagatccagtatttggcaatcaaattattccggatacaactatactatgtgctgttccattccatcatgcgtttggcactttcacaaatttaggatatttaatatgtggcttccacgtagtgcttatgtacagattcaatgaacatttattcttacaaacactacaagattacaaatgtcaaagcgcgttactagtacctacagtacttgcgtttcttgctaaaaaccctttggttgataaatatgatttatcaaatttacatgaaattgcttctgggggtgccccactttcaaaagaaatttcagaaatagcagcaaaacgatttaaactaccaggaatacgacaagggtatggtctaactgaaacaacgtgtgctattgtaattactgctgaaggagaatttaaacttggggctgtcggaaaagttgtaccattttattccttaaaagttcttgatcttaatacaggaaaaaaattggggccaaacgagaggggggaaatatgtttcaaaggacctatgattatgaaaggttatataaataatccagaagcaacacgcgagttaattgatgaagagggatggatacactctggtgatataggatattttgatgaagatggccatgtatacattgttgatcgattgaaatctttgattaaatacaaaggctatcaagttccgcccgccgagttagaagctttactgctgcagcatccgtttattgaagatgcaggagttgcgggtgttcccgatgaagttgcgggtgatcttcctggtgctgttgtagttttaaaagaaggaaaatctattacagaaaaagaaatccaagattacgtggcaggccaagtaacttcttcgaaaaagttacgaggaggtgttgaatttgtgaaagaggtacccaaaggttttactggaaaaattgataccagaaaaataaaagaaatacttattaaggcacaaaaaggcaaatccaaatccaaagccaaattgtaaactaagtgtttgttaatgttgttaaacattttataaaatacactgtagctatttattagtaaccaaaatgcttctaacatcaagatgcctatatctaagaacgttgtatttatatactttggggtttttggtgattatgtcaaatgtatgtgtgaaaagggtatacgtagtttaagggacataaaaataaataaaattaattattggatttgg